(19) United States
(12) Patent Application Publication
(10) Pub. No.: US 2008/0102135 A1
(43) Pub. Date: May 1, 2008

(54) Use of equine amniotic membrane in ophthalmic surgeries in veterinary medicine

(76) Inventor: Franck Jean Ollivier, Meylan (FR)

Correspondence Address: Franck Ollivier, Unit #12, 607 South Albany Avenue, Tampa, FL, 33606, US

(21) Appl. No.: 11/554,019

(22) Filed: Oct. 28, 2006

(51) Int. Cl. A61K 35/54

(52) U.S. Cl. 424/582

(57) ABSTRACT

A method for making, storing and using a surgical graft from equine amniotic membrane in veterinary ophthalmology. The amniotic membrane is obtained from equine placenta, from which the chorion has been separated. Sheets of the amniotic membrane are cut to size and mounted on filter paper. The cells of the amniotic membrane are killed, preferably while being frozen and thawed in the storage solution. The equine amniotic membrane can be used in a variety of ocular surgeries in horses but also other species such as food animals, dogs and cats. It represents a strong biomaterial that will give a good physical support to the ocular tissues while inducing a minimal amount of scarring which is primordial in ocular surgeries in order to obtain the best visual outcome.

### BACKGROUND OF THE INVENTION

In pets as well as in food animals and even more in horses, corneal ulcerations are very common; most of them are uncomplicated non-infected ulcers that heal quickly without complications but some can present various levels of corneal liquefaction or “melting”. Melting corneal ulcers are induced by an important corneal proteolytic activity, they can progress rapidly and be sight threatening. The treatment of melting corneal ulcers should eradicate the infection, reduce or stop the corneal destruction, support the corneal integrity, and minimize corneal scarring.¹⁻⁸ They represent a medical and /or surgical challenge for the veterinary ophthalmologists.

The removal of large neoplastic and or necrotic lesions on the conjunctiva/sclera/cornea in pets, food animals and horses can be very challenging such as corneolimbal squamous cell carcinomas, limbal melanomas, and corneal sequestrum.^{1,3,4} It can be a difficult challenge because of the lack of tissue to cover the defect left by the mass removal but also to keep the eye visual.

Amniotic membrane (AM) consists of an epithelium, a thick basement membrane, and an avascular stroma. High concentration of basic fibroblast growth factor and basement membrane components like collagen are present. It provides a good cell-basement membrane structure that is critical for epithelial proliferation and differentiation.⁹

It is a strong biomaterial that contains anti-angiogenic, anti-inflammatory, anti-fibrotic and growth factors. A recent investigation revealed that AM also contains several proteinase inhibitors such as α2-macroglobulin, α1-chymotrypsin inhibitor, interα-1-trypsin inhibitor, α1-trypsin inhibitor, α2-plasmin inhibitor.¹⁰⁻¹¹

Reports have shown interest in the human AM as a graft for reconstruction of various ocular surfaces in humans and rabbit.¹²⁻¹⁷ However, there is no information on the use of the equine amniotic membrane for ophthalmic surgeries in horses as well as in pets, and food animals.

### BRIEF SUMMARY OF THE INVENTION

For the first time, I describe the harvest, making, storage and use of the equine amniotic membrane for ophthalmic surgeries in horses but also food animals, dogs and cats. I think there are tremendous benefits to use of the equine amniotic membrane in veterinary ophthalmology. The equine amniotic membrane is very easy to harvest, to separate from the other placental envelops, to store and to use. I have identified many ocular diseases in horses, food animals, dogs and cats that can be treated surgically with the use of the equine amniotic membrane as mentioned in the background paragraph and detailed below.

Melting ulcers are a very common corneal disease in animals and they represent a true medical and/or surgical challenge in order to save the eye and to obtain minimal amount of scarring for the best visual outcome. Corneal proteinases induce these melting ulcers. Therefore, control of the proteolytic activity and support of the cornea are primordial in the treatment of these ulcers.⁸⁻²⁰ The therapeutic effect of AM on severely damaged melting ulcers is due to the inhibitory effect of AM on corneal proteinase activity that would otherwise induce severe and irreversible stromal destruction. It has also been suggested that the amniotic membrane can decrease the protease activity directed against the corneal stroma by providing exogenous collagen as a deviant substrate for the tear film proteases besides being a strong physical support for the melting cornea.^{5-8,21} The anti-inflammatory, anti-fibrotic and anti-angiogenic agents present in the AM will also have a important role in reducing the amount of scarring. Therefore, the equine amniotic membrane has use in eye problems in pets, food animals as well as in horses and particularly to surgically treat melting corneal ulcers.

There are also many ocular diseases in veterinary ophthalmology where surgery is a real challenge either because of the lack of tissue to work with or because of the difficulty to have the best visual outcome.^{1,3,4} Comeolimbal squamous cell carcinomas, corneal sequestrum or any other scleral/corneal/conjunctival neoplastic or necrotic lesions in pets, food animals, and horses represent a very good indication for the use of the amniotic membrane. The equine amniotic membrane is a tissue readily available to cover any scleral/corneal/conjunctival defect after the removal of a neoplastic or necrotic lesions, particularly if the lesions are multiple or large. Another advantage to use the equine amniotic membrane is that the membrane contains anti-inflammatory, anti-fibrotic and anti-angiogenic agents and the amniotic membrane graft is nicely incorporated into the corneal/conjunctival/scleral defect and seems to create noticeably much less scarring than any other grafting material.²²

### DETAILED DESCRIPTION OF THE INVENTION

The harvest and the use of the human amniotic membrane has been previously reported. However, nobody prior to my application has reported the harvest, making, storage and use of the equine amniotic membrane for ophthalmic surgeries in horses as well as dogs, cats and food animals.

The equine placenta is harvested during an elective cesarean section for term pregnancy on a mare with no infectious disease. The allantoammnion is separated from the allantochorion and the amnion (epithelium, stroma and basement membrane) is then separated by blunt dissection from the allantois. The AM was then placed on a 0.45 μm pores nitrocellulose paper (with the allantoic (stroma) side against the paper) and stored frozen at −80° C. for years in Dulbecco's modified Eagle medium and glycerol at ratio of 1:1 (volume to volume), also containing antibiotics and antifungal (penicillin 50 μg/mL, streptomycin 50 μg/mL, neomycin 100 μg/mL, and amphotericin B 2.5 μg/mL). After the storage in the freezer, the AM epithelium is almost inexistent therefore the AM graft material consists of a stroma and a basement membrane.

It is very difficult to separate the amnion from the other placental envelops in dogs and cats and the canine or feline amniotic membrane is not as strong as the equine amniotic membrane and therefore does not represent a good biomaterial to use in ophthalmic surgeries. Because the equine amniotic membrane stored frozen does not contains a lot of cells, if any, it is a biomaterial that can be used not only in horses but in other species such as dogs, cats and food animal as it will not induce any severe foreign body type or immune reaction.

Prior to ophthalmic surgery, the AM is naturally thawed, and then rinsed with sterile saline during 30 minutes to wash off the glycerol. During the wash, the AM is kept on the nitrocellulose paper with the allantoic (stroma) side against the paper. The amniotic membrane is then prepared and cut according to the size of the defect to cover. Amniotic membrane grafts should simply cover the defect with little tension present before the suture placement to reduce premature graft retraction. Like for any grafts, it is important that the graft site is properly prepared. The recipient bed for the amniotic membrane graft is prepared by removing loose epithelium and necrotic corneal tissues with a Beaver No. 64 microsurgical blade.

The equine amniotic membrane can be in many ophthalmic surgeries in pets, food animals and horses where a defect needs to be covered: corneal ulcers, corneal perforations, defects left after removal of scleral/conjunctival/corneal necrotic or neoplastic lesions. It is a strong material, easily available, in large size, that contains anti-angiogenic, anti-inflammatory, anti-fibrotic and growth factors. For these reasons the equine amniotic membrane represents an ideal biomaterial to use in ophthalmic surgeries in dogs, cats, food animals and horses.

The purpose and the evolution of the AM graft depends on how the AM is placed on the defect site: if the allantoic (stromal) side of the AM faces the defect, the AM graft will adhere to the defect and the AM is expected to be incorporated to the tissue; on the other hand, if the basement membrane of the AM faces the defect, the tissue epithelial cells will migrate along the membrane and therefore the AM is used as a bandage. It is expected to slough off in 7-10 days.

The amniotic membrane is then sutured to the graft site with simple interrupted or continuous sutures of 7-0 to 8-0 absorbable suture material. Several layers of amniotic membrane can be used and piled up to fill the defect if necessary. The AM may be placed limbus to limbus to cover the complete cornea as a bandage. A temporary tarsorrhaphy can be then placed to minimize blinking movement, prevent excessive lid trauma to the graft and its sutures, and to allow quick graft adherence to the defect site.

### REFERENCE

1. Brooks D E: Equine ophthalmology, in Gelatt K N (ed): Veterinary Ophthalmology (ed 3). Philadelphia Pa., Lippincott Williams & Wilkins, 1999, pp 1053-1116
2. Brooks D E: Corneal ulceration, in Brooks D E (ed): Equine ophthalmology for equine practitioner (ed 1). Jackson W G, Teton Newmedia, 2004, pp 58-87
3. Severin G A (ed): Veterinary ophthalmology notes, (ed 2). Fort Collins, Colo., Colorado State University, 1976
4. Gilger B C, Whitley R D: Surgery of the cornea and sclera, in Gelatt K N (ed): Veterinary Ophthalmology (ed 3). Philadelphia Pa., Lippincott Williams & Wilkins, 1999, pp 675-754
5. Ollivier F J, Gaillard-Lavirotte C. Identifier et traiter les ulcerations corneenes a collagenases chez les equides. Le Nouveau Practicien Veterinaire Equin. 2006;9:22-28
6. Ollivier F J, Medical and surgical management of melting corneal ulcers exhibiting hyperproteinase activity in the horse. Clinical techniques in equine practice 2005 Mar ;4(1):50-71.
7. Ollivier F J. Proteases. In Andrew S E, Willis A M, Ollivier F J, Mathews A. Diseases of the cornea and sclera, in Equine Ophthalmology (Gilger B C Ed.) Elsevier Saunders, St Louis, Mo., 2005.
8. Ollivier F J, Bacterial corneal diseases in dogs and cats. Clinical techniques in small animal practice, 2003; 18: 193-198.
9. Kim J S, Kim J C, Na B K, et al. Amniotic membrane patching promotes healing and inhibits proteinase activity on wound healing following acute corneal alkali burn. Exp Eye Res 70: 329-337, 2000
10. Hao Y, Ma D H, Hwang D G, et al. Identification of antiangiogenic and antiinflammatory proteins in human amniotic membrane. Cornea 19: 348-352, 2000
11. Fortunato S J, Menon R, Lombardi S J. Collagenolytic enzymes (gelatinases) and their inhibitors in human amniochorionic membrane. Am J Obstet Gynecol 177: 731-741, 1997
12. Shimazaki J, Yang H Y, Tsubota K. Amniotic membrane transplantation for ocular surface reconstruction in patients with chemical and thermal burns. Ophthalmology 104: 2068-2076, 1997
13. Tseng S C, Prabhasawat P, Lee S H. Amniotic membrane transplantation for conjunctival surface reconstruction. Am J Ophthalmol 124: 765-774, 1997
14. Azuara B A, Pillai C T, Dua H S. Amniotic membrane transplantation for ocular surface reconstruction. Br J Ophthalmol 83: 399-402, 1999
15. Kim J C, Tseng S C. Transplantation of preserved human amniotic membrane for surface reconstruction in severely damaged rabbit corneas. Cornea 14: 473-484, 1995
16. Kruse F E, Joussen A M, Rohrschneider K, et al. Cryopreserved human amniotic membrane for ocular surface reconstruction. Graefes Arch Clin Exp Ophthalmol 238: 68-75, 2000
17. Kruse F E, Rohrschneider K, Volcker H E. Multilayer amniotic membrane transplantation for reconstruction of deep corneal ulcers. Ophthalmology 106: 1504-1510, 1999
18. Ollivier F J, Brooks D E, Kallberg M E, Komaromy A M, Lassaline M E, Andrew S E, Gelatt K N, Stevens G R, Blalock T D, van Setten G, Schultz G S. Evaluation of various compounds to inhibit activity of matrix metalloproteinases in the tear film of horses with ulcerative keratitis. American Journal of Veterinary Research. 2003, 64: 1081-1087.
19. Brooks, D E, Ollivier F J, Matrix metalloproteinase inhibition in corneal ulceration. Veterinary Clinics North America Small Animal Practice, 2004; 34(3):611-22.
20. Ollivier F J, The precorneal tear film in horses: its importance and disorders. Veterinary Clinics North America Equine Practice, 2004; 20(2):301-18.
21. Lassaline M E, Brooks D E, Ollivier F J, Komaromy A M, Kallberg M E, Gelatt K N. Equine amniotic membrane transplantation for corneal ulceration and keratomalacia in three horses. Vet Ophthalmology. 2005 Sep-Oct;8(5):311-7.
22. Ollivier F J, Kallberg M E, Plummer C E, Barrie K P, S O'Reilly, D Taylor, Gelatt K N, Brooks D E. Amniotic membrane transplantation for corneal surface reconstruction after excision of corneolimbal squamous cell carcinomas in 9 horses. 2006;9:404-413.

1. The equine amniotic membrane represents a very good biomaterial that can be used in many ophthalmic surgeries performed in dogs, cats, food animals and horses. It is a strong material that will give a very good support to the ocular tissues and therefore can be used to cover any defects. The amniotic membrane contains anti-angiogenic, anti-inflammatory, anti-fibrotic and can either slough off from the graft site after healing (used as a bandage) or can be nicely incorporated to the graft site leaving minimal amount of scar and therefore the best visual outcome possible which is an main objective when performing ocular surgeries in animals.
